(19) United States
(12) Patent Application Publication
(10) Pub. No.: US 2008/0119786 A1
(43) Pub. Date: May 22, 2008

(54) Hypodermic Syringe

(75) Inventors: Daniel Craig Stewart, Newstead (AU); Robert Warring Geddes, Newstead (AU); Roy Tudor Brewer, Gayndah (AU); Craig Wilson, Bondi Junction (AU)

Correspondence Address: Stephen E Reiter;Foley & Lardner, P O Box 80278, San Diego, CA, 92138-0278, US

(73) Assignee: BREWER RETRACTABLE TECHNOLOGIES PTY LTD, MAPLETON, QUEENSLAND AUSTRLIA (AU)

(21) Appl. No.: 10/582,734

(22) PCT Filed: Dec. 20, 2004
(86) PCT No.: PCT/AU04/01793

§ 371 Date: Jul. 18, 2007

(30) Foreign Application Priority Data
Dec. 18, 2003 (AU) 2003907029

(51) Int. Cl. A61M 5/50

(52) U.S. Cl. 604/110

(57) ABSTRACT

A syringe (110) having a barrel (111) that receives a rod assembly (117). Mounted at the forward end of the Barrel (111) is a needle mounting (121) that receives as needle (124). The mounting (121) is releasably attached to the barrel (111). The rod assembly (117) includes a hollow rod (118) that has at its forward end a gripper device (135) that engages the mounting (121) so that upon release of the mounting (121) with respect to the barrel (111) and release of the device (135) with respect to the rod (118), the mounting (121) and device (135) are propelled into the rod (118) by a spring (138).

### TECHNICAL FIELD

The present invention relates to the hypodermic syringes and more particularly to hypodermic syringes that retract a needle to the interior of the syringe after use.

### BACKGROUND OF THE INVENTION

The safe disposal of sharp medical instruments is of a prime concern to health care professionals such as doctors and nurses. For example, a particular problem is the safe disposal of needles. An accidental puncture can result in the health care professional contacting a serious disease such as Acquired Immune Deficiency Syndrome and Hepatitis.

A wide variety of methods are proposed to inhibit accidental needle injuries including withdrawing the needle after use into the interior of the syringe. Such arrangements are described in International Patent Publications WO92/18186, WO91/10461 and WO01/17594 as well as U.S. Pat. Nos. 5,000,736 and 5,125,898.

Although it is desirable to withdraw the needle into the interior of the syringe after use, such constructions are often complex and difficult to manufacture. The devices described in the abovementioned patent publications suffer from a number of disadvantages, the disadvantages including the cost and difficulty of manufacture and in some instances reliability of the mechanism within the syringe that retracts the needle into the interior of the body of the syringe.

More particularly two devices of the above described patent publications withdraw the needle into the interior of the hollow plunger by means of a reduced air pressure within the plunger.

### OBJECT OF THE INVENTION

It is the object of the present invention to overcome or substantially ameliorate at least one of the above disadvantages.

### SUMMARY OF THE INVENTION

There is disclosed herein a syringe having a longitudinal axis, a forward end with a needle, and a rearward end, said syringe including:

a barrel providing a cylindrical bore;

a needle mounting to which said needle is fixed so as to extend forwardly therefrom;

a piston rod assembly slidably received in said bore and in sealing contact therewith so as to co-operate with said bore to provide a variable volume chamber to receive a liquid to be injected, said assembly including;

a hollow rod extending rearwardly from within said barrel to enable a user to move said assembly to various said volume, said rod having a cavity extending rearwardly from a forward opening in said rod;

a gripper device mounted at said forward opening; said syringe further including:

a gripper retainer extending between the device and rod to maintain said device fixed to said rod and movable to release said device so that said device moves into said cavity upon said gripper retainer moving forward at said fore end; and wherein

said mounting closes said chamber with said needle communicating therewith so that upon a reduction in volume of said chamber said liquid is forced through said needle, said mounting including:

a body engaged by said gripper device when adjacent said forward end and before said retainer releases said device, said body when engaged by said device is fixed thereto;

means to urge said body and device in to said cavity; and wherein

said mounting includes a mounting retainer securing said mounting to said barrel but operable to release said mounting so that said mounting moves together with said needle, with said device into said cavity, said mounting retainer being radially moved inward from a retaining position to a release position by forward movement of said piston rod; and

an actuation member moved longitudinally forward by the forward movement of said piston rod to thereby actuate said mounting retainer to move radially inward to release said mounting after engagement of said mounting with said device.

Preferably, the means to urge includes said gripper device closing said opening so that said cavity can maintain a reduced internal pressure relative to atmosphere, so that upon said device engaging said body and said device and mounting being released, said device mounting and needle are moved into said cavity.

In an alternative preferred form, said means to urge includes a spring engaging said mounting and urging said mounting and device into said cavity.

Preferably, the gripper retainer engages a portion of said barrel at said forward end to release said device.

Preferably, said gripper retainer has an engaging portion that upon complete of the injection stroke attaches the rod to the barrel to prevent rearward movement of the rod.

Preferably, the mounting includes a rearwardly extending projection, and said gripper device includes a cavity to receive said projection with said projection and gripper device engaging to captively locate said projection in said cavity.

Preferably, said gripper device includes a neck extending to said cavity, with said projection passing through said neck, with said projection including an expansion member that contracts as projection passes through said neck and expands to captively locate the projection in said cavity.

Preferably, said mounting includes a forward portion to sealingly connect the mounting to the barrel.

Preferably, said syringe includes a cap at the forward end of said barrel, which cap receives said mounting and said forward portion.

Preferably, said cap includes at least one passage to allow air to enter the cap so that air pressure is applied to the mounting to urge the mounting into the rod cavity.

Preferably, said actuator member is a sleeve surrounding said mounting.

Preferably, said mounting retainer includes a plurality of fingers that are resiliently urged outwardly, and are engaged by a ramp surface of said actuator member to be moved radially inwardly to release the mounting.

Preferably, said cap includes a forwardly facing abutment surface engaged by said fingers.

Preferably, said spring is located in said cap and engages said cap and mounting so as to be compressed therebetween to urge said mounting and device into said cavity.

There is disclosed herein a syringe having a longitudinal axis, a forward end to which a needle is to be attached, and a rearward end, said syringe including:

a barrel providing a cylindrical bore;

a needle mounting to which said needle is to be attached so as to extend forwardly therefrom, said mounting being at said forward end and providing for the delivery of a liquid to be injected to the needle;

a piston rod assembly slidably received in said bore and in sealing contact therewith so as to co-operate with said bore to provide a variable volume chamber to receive the liquid to be injected, the chamber being located between said rod assembly and said forward end, said piston rod assembly including:

a piston rod extending rearwardly from within said barrel to enable a user to move said assembly to vary said volume, said rod having a cavity extending rearwardly from a forward opening in said piston rod;

a gripper device mounted at said forward opening and having a stem projecting towards said forward end,

a resilient member located in said rod and attached to said rod and device and urging the device into said cavity,

a gripper retainer extending between the device and rod to maintain said device fixed to said rod and operable to release said device so that said device moves into said cavity upon said gripper retainer moving forward at said forward end; and wherein

said mounting closes said chamber so that upon a reduction in the volume of said chamber said liquid is forced through said needle, said mounting including:

a body engaged by said gripper device when adjacent said forward end and before said retainer releases said device, said body when engaged by said device being fixed thereto, said body having a passage into which said stem is to project to fix the body to the gripper device;

a mounting retainer securing said mounting to said barrel but operable to release said mounting so that said mounting moves, together with the needle when attached thereto, with said device into said cavity, said mounting retainer being moved inwardly relative to said axis from a retaining position to a release position by forward movement of said piston rod; and wherein

said syringe further includes:

an actuation member moved longitudinally forward by the forward movement of said piston rod to thereby actuate said mounting retainer to move inwardly to release said mounting after engagement of said mounting with said device.

Preferably, said resilient means is a spring urging said device into said cavity.

Preferably, said spring is compressed to urge the device into said cavity.

Preferably, said spring is tensioned towards the device into said cavity.

Preferably, said spring extends between a rearward portion of said device and a rearward portion of said plunger.

Preferably, said actuation member is part of said rod assembly.

Preferably, said actuation member is fixed to said rod.

Preferably, said mounting includes a forward portion to sealingly connect the mounting to the barrel.

Preferably, said actuation member is a sleeve surrounding said stem.

Preferably, said mounting retainer includes a plurality of fingers that are resiliently urged outwardly relative to said axis, and are moved inwardly relative to said axis to release said mounting.

Preferably, said mounting includes a catch to engage said stem when said stem projects into said mounting.

Preferably, said stem has an enlarged forward extremity that is engaged by said catch.

Preferably, said gripper retainer is moved inwardly with respect to said axis to release said device.

Preferably, said gripper retainer engages said barrel to be moved inwardly with respect to said axis.

Preferably, said gripper retainer is a circlip that is resiliently deformed by said barrel to be moved inwardly with respect to said axis.

Preferably, said gripper device projects inwardly of said rod and is slidably guided thereby for movement between a forward position to engage said mounting, and a retracted position located within said rod together with said mounting.

Preferably, said spring extends between a rearward portion of said device and a forward portion of said rod.

### BRIEF DESCRIPTION OF THE DRAWINGS

Preferred forms of the present invention will now be described by way of example with reference to the accompanying drawings wherein:

FIGS. 1 to 8 schematically depict in sectioned side elevation a syringe in various operative configurations;

FIG. 9 is a schematic sectioned side elevation of a forward portion of the syringe of FIG. 1;

FIG. 10 is a schematic sectioned side elevation of the forward portion of FIG. 9 in a further configuration;

FIG. 11 is a schematic sectioned side elevation of the forward portion of FIG. 9 in a still further configuration;

FIG. 12 is a schematic sectioned side elevation of a modification of the syringe of FIGS. 1 to 11;

FIG. 13 is a schematic sectioned side elevation of the syringe of FIG. 12 in a further operative position;

FIG. 14 is a schematic sectioned side elevation of the syringe of FIG. 12 in a further operative configuration;

FIGS. 15 to 17 are schematic sectioned side elevations of a further hypodermic syringe;

FIGS. 18 to 20 are schematic sectioned side elevations of a modification of a syringe;

FIGS. 21 to 25 are each a schematic sectioned side elevation of the forward portion of a syringe; and

FIG. 26 is a schematic parts exploded isometric portion of a syringe having the forward portion of FIGS. 21 to 25.

### DETAILED DESCRIPTION OF THE PREFERRED EMBODIMENTS

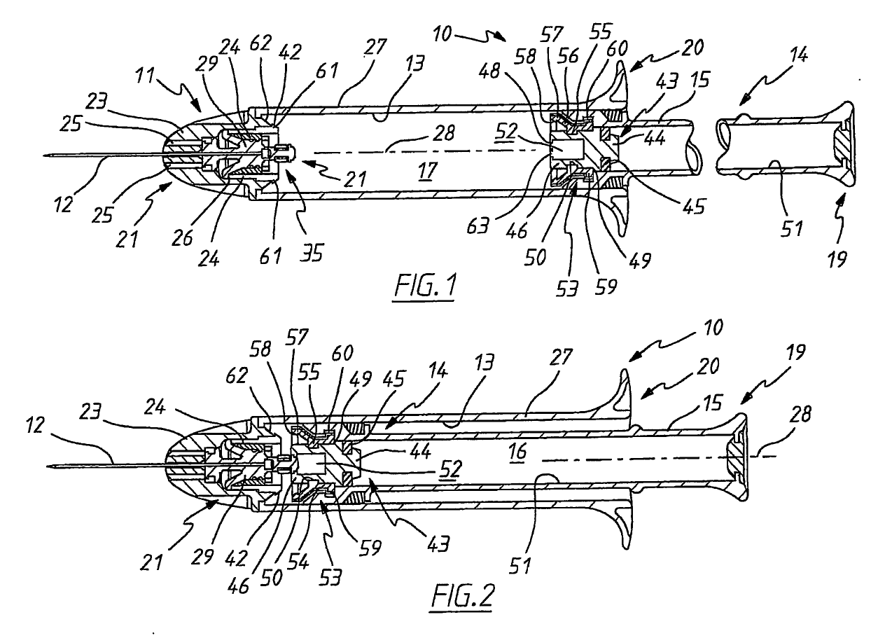

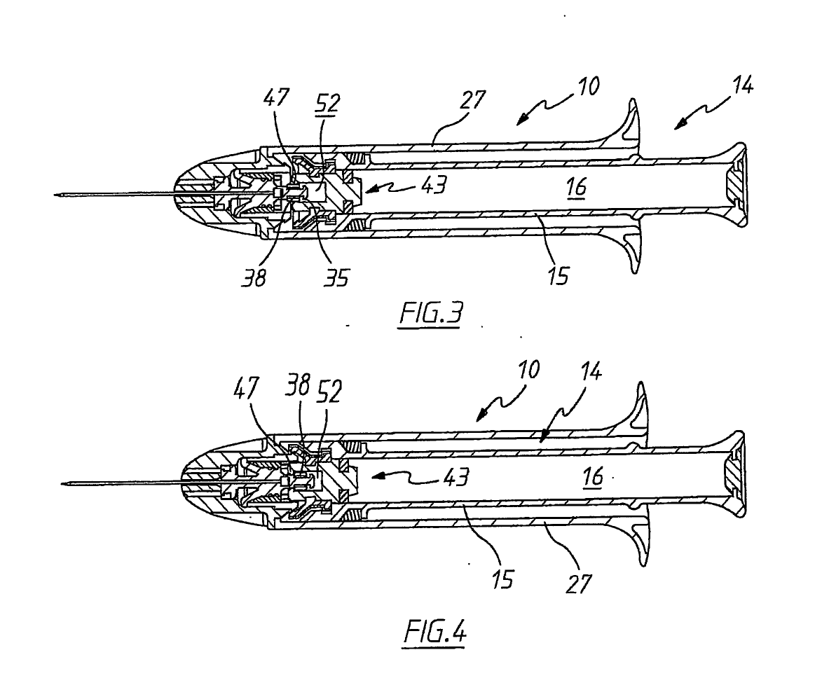

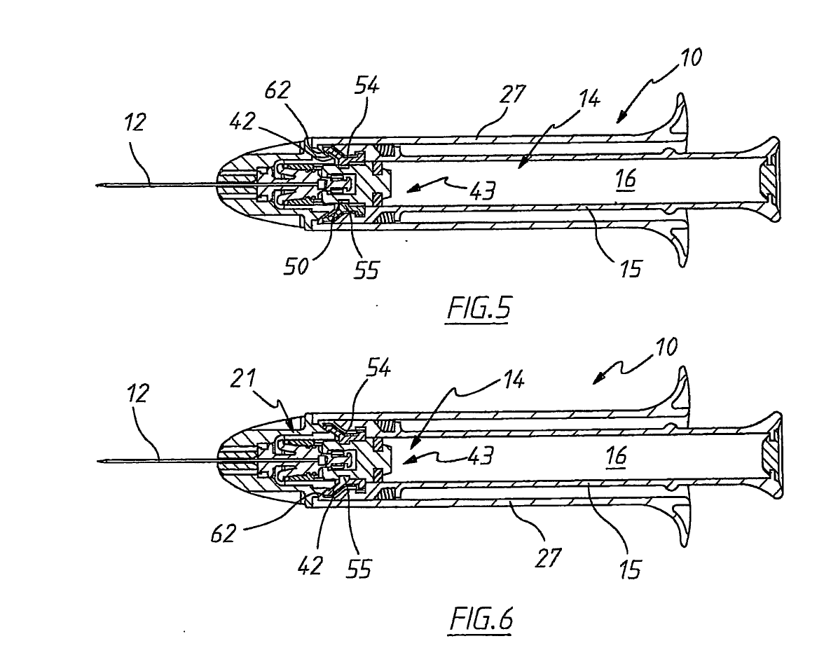

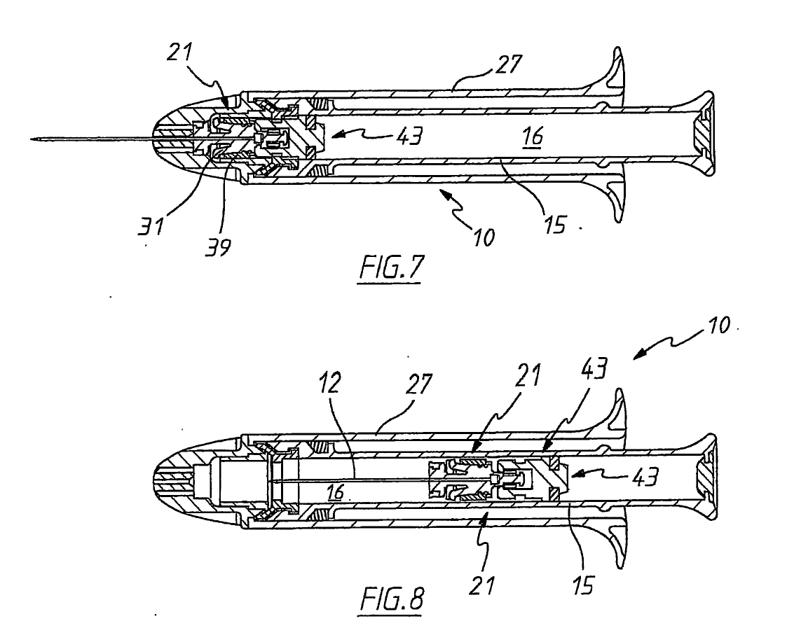

In FIGS. 1 to 11 of the accompanying drawings there is schematically depicted a hypodermic syringe 10. The syringe 10 includes a forward portion 11 that supports a needle 12. The syringe 10 includes a barrel 27 with an internal cylindrical bore 13. Slidably received within the bore 13 and in sealing contact therewith is a piston rod assembly 14 including a hollow rod 15 enclosing a cavity 16. The assembly 14 co-operates with the bore 13 to enclose a variable volume chamber 17 that receives a liquid to be injected. The chamber 17 communicates with the hollow needle 12, with a reduction in the volume of the chamber 17 causing the liquid to be injected through the interior of the needle 12.

The assembly 14 includes seals 18 that contact the bore 13 to sealingly connect the assembly 14 to the barrel 12. The rearward end 19 projects outwardly from within the barrel 27 and splays outwardly so that a user can quip the rearward end 19 and cause movement of the assembly 14 to change the volume of the chamber 17.

The barrel 12 also has a rearward end 20 that is also splayed outwardly to facilitate a user gripping the syringe 10.

Closing the forward end of the chamber 17 is a needle mounting 21. More particularly the needle 12 is fixed to the mounting 21 so as to extend forwardly therefrom through a passage 22 in the end cap 23. The end cap 23 is attached to the forward extremity of the barrel 12 and provides a sleeve 24 that receives the mounting 21. The end cap 23 further includes passages 25 through which air may pass.

The sleeve 24 is fixed to the barrel 27 and includes a forwardly facing abutment surface 26 that is generally radially extending relative to the longitudinal axis 28.

The mounting 21 includes a body 29 having radial passages 30 that communicate with the inner extremity of the needle 12, with the passages 30 communicating with the chamber 17 to provide for the flow of liquid from the chamber 17 to the needle 12. The body 27 has extending from its forward end a plurality of resilient fingers 31 that are angularly spaced about the axis 28. The fingers 31 are resiliently urged radially outward so as to engage the surface 26 to retain the mounting 21 within the sleeve 24, that is fixed with respect to the barrel 27. The fingers 31 provide a needle mounting retainer to retain the mounting 21 in position during normal operation of the syringe 10.

Attached to the forward end of the body 21 is a sealing stem 32 having a seal 33 that engages a cylindrical surface 34 to sealingly connect the mounting 21 with the cap 23 and therefore sealingly close the forward portion of the chamber 17.

Extending rearwardly from the mounting body 29 is a projection 35 with a head 36 from which there extends a stem 37. The head 36 is enlarged radially relative to the stem 37 with respect to the axis 28.

The body 29, fingers 31, projection 35 and stem 32 are integrally formed from plastics material so that the fingers 31 are resiliently deformable so as to be movable radially inwardly and yet be urged outwardly to engage the surface 26.

Located around the stem 37 is a “split” ring 38 that is formed from resilient plastics material and when in a relaxed configuration is retained captive on the stem 38 by having an inner diameter less than the outer diameter of the head 36. However, the ring 36 is resiliently deformable so as to have its overall outer diameter decreased.

Surrounding the body 29 and captively located with respect thereto is an actuation member 39 which is of a cylindrical configuration so as to have an outer cylindrical surface in sliding contact with the internal surface of the sleeve 24 while having an inner ramp surface 40 which tapers in radius rearwardly so that the member 39 acts as a wedge so that upon sliding engagement with the fingers 31 during forward movement of the member 39, the fingers 31 are caused to resiliently deflect radially inward to disengage from the surface 26.

As can be noted, the fingers 37 have radially outer surfaces 41 that are inclined relative to the axis 18 so as to co-operate with the surface 40 to cause the fingers 31 to deflect inwardly.

The sleeve 24 has a frusto-conical rear ramp surface 42. The surface 42 has as its longitudinal axis the axis 28 and tapers rearwardly.

In respect of the above mounting 21 it should be appreciated that it is releasable from within the sleeve 24 by radial inward deflection of the fingers 31. With the body 29 having fixed to it the needle 12, rearward movement of the body 29 takes with it the needle 12.

The assembly 14 includes a gripper device 43 that includes a body 44 with a seal 45 that closes the forward opening 72 and therefore the cavity 16 so that the cavity 16 can maintain a reduced pressure. Accordingly the pressure within the cavity 16 is less than ambient air pressure surrounding the syringe 10. The body 44 has extending forwardly from it an annular flange 46 that terminate with a radially inwardly extending annular lip or projections 47 providing a neck 63. The lip 47 or each projection is provided with a forwardly facing inclined annular ramp surface 48. The body 44 has a radially extending generally circular surface 49 as well as a generally annular recess 50. The body 44 is adapted to be receivable within the cavity 16. That is the device 43 has a diameter less than the diameter of the cylindrical surface 51 of the rod 15 surrounding the cavity 16.

The fingers 46 surround a cavity 52 to accommodate the projection 35 when the device 43 is at its forward extremity in respect of movement.

Mounted on the device 43 is a gripper retainer 53 in the form of a ring 54. The ring 54 has an annular ridge 55 that projects inwardly of the recess 50, and has a forwardly extending frusto-conical flange 56.

The flange 56 terminates with a radially inwardly extending annular barb 57 as well as a ramp surface 58, which ramp surface 58 is to co-operate with the ramp surface 42 of the sleeve 24, and ramp surface 62 behind the barb.

In operation of the above described syringe 10, the device 43 starts at a forward position, which forward position has the ring 38 spaced forwardly of the lip 47 so as to not be captively located with respect to the body 44. The rod 15 is moved rearwardly so as to draw a liquid into the chamber 17 due to the chamber 17 increasing in volume. Once a desired volume of liquid is retained within the syringe 10, the rod 15 is moved forwardly to cause a reduction in the volume of the chamber 17. The liquid is forced out of the chamber 17 through the passages 30 to exit via the hollow needle 12 and more particularly the extremity thereof. When the stroke of the rod 15 is completed, the device 43 engages the mounting 21 with the result that the device 43 and mounting 41 together with the needle 12 are withdrawn into the cavity 16 due to air pressure being applied to the mounting 41 and body 44. The mounting 21 and body 44 together with needle 12 are pushed into the cavity 16 so that the needle 12 is no longer exposed.

More particularly forward movement of the rod 15 beyond a predetermined position causes the projection 35 to enter the cavity 52 via the neck 63. Location of the projection 35 in the cavity 52 locates the ring 38 rearwardly of the lip 47 so that the projection 35 is captively located within the cavity 52. The ring 38 is caused to contract radially due to its sliding engagement with the ramp surface 48. This allows the projection 35 to enter the cavity 52. Once past the lip 47 the ring 38 radially expands so that it captively locates the projection 35 with respect to the body 44. Further forward movement of the body 44 causes the fingers 46 to abut the member 39 to also cause it to slide longitudinally forward. Engagement of the surface 40 with the surfaces 41, as the member 39 moves forward, results in the fingers 31 moving radially inward so as to clear the surface 26. This action releases the mounting 21 to move with the device 43. Thereafter further forward movement of the device 43 causes the flange 56 to radially expand due to engagement of the surface 58 with the surface 42 of the sleeve 24. Continued radial expansion of the flange 56 moves the ridge 55 from within the recess 50 so that the device 43 is now released from the rod 15.

Upon release of the mounting 21 and release of the device 43, the mounting 21, device 23 and needle 12 are pushed by air pressure into the cavity 16. Air is allowed to enter the cap 23 via passages 25 so that air pressure is applied to the mounting 21 and device 43.

In respect of the ring 54 it should be appreciated that it has an annular ridge 59 that engages within an annular recess 60 in the rod 15 so that the ring 54 moves therewith and will not move rearwardly with the device 43. Still further, the annular barb 57 engages an annular barb 61 of the sleeve 24 so that the ring 54 becomes attached to the sleeve 54 and therefore the barrel 27 to retain the rod 15 in its forward most position upon completion of the injection stroke.

In FIG. 1 the syringe 10 is depicted with the piston rod fully retracted so as to maximise the volume of the chamber 17 containing a liquid to be injected. Thereafter the syringe 10 is operated to move the piston rod assembly 14 toward the needle 12. This forces the liquid out through the needle 12. Toward the end of its stroke, the gripper device 14 engages the projection 35 as shown in FIG. 2. Further forward movement of the piston rod assembly 14 causes contraction of the ring 38, as shown in FIG. 3. The piston rod assembly 14 further progresses until the ring 38 is contained in the cavity 52 as shown in FIG. 4, with the ring 38 expanded and therefore captively located with respect to the gripper device 43. Further forward movement of the piston rod assembly 14 results in the ring 54 engaging the surface 42 to the extent that the annular ridge 55 leaves the annular recess 50, thereby releasing the device 43. This is shown in FIG. 5. The barb 57 then engages behind the barb 61 so the piston rod assembly 14 is captively located in the forward position, as shown in FIG. 6. Further forward movement of the assembly 14 causes the actuation member 39 to slide forward to thereby radially retract the fingers 31, as shown in FIG. 7. With the mounting 21 now released due to continued engagement of the barb 57 with the surface 62, the mounting 21 together with the device 43 are drawn back into the piston rod 15, as shown in FIG. 8.

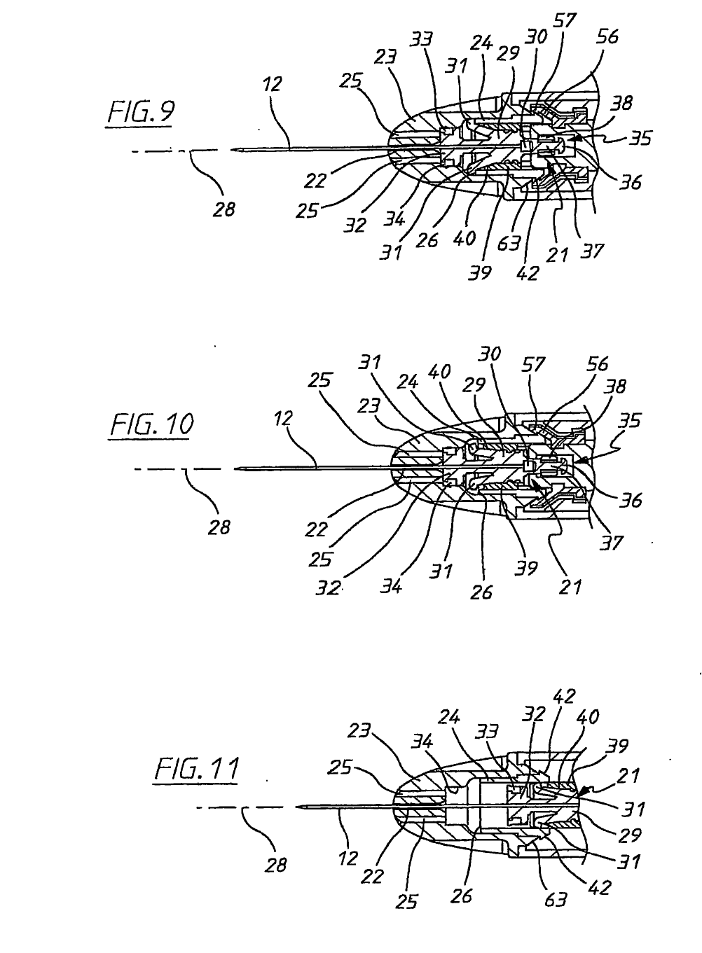

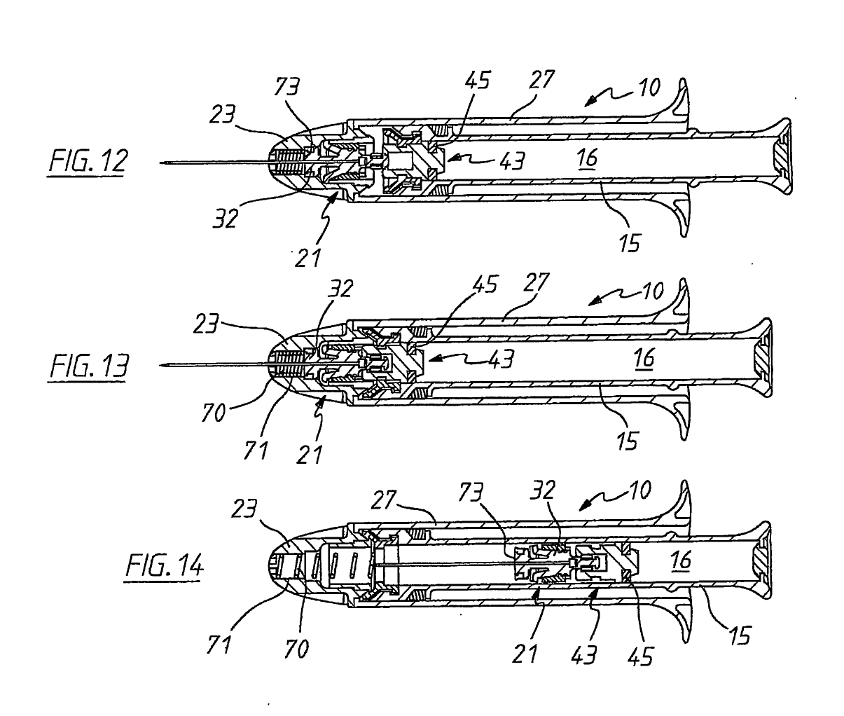

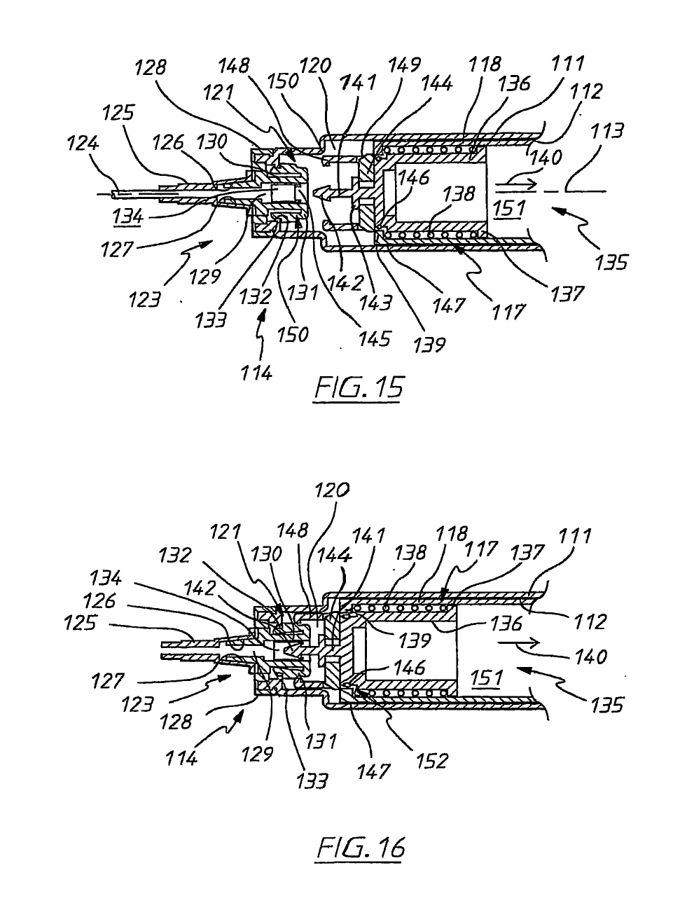

In the embodiment of FIGS. 12 to 14, the syringe 10 depicted has been allocated the same reference numerals as the previous embodiment. However, in this embodiment you will note the absence of seal 45 as the chamber 16 is no longer required to maintain a reduced internal pressure.

To urge the gripper device 43 together with the mounting 21 attached thereto into the cavity 61 there is provided a spring 70 having a forward end mounted in the cap 23. More particularly the cap 23 has a passage 71 that contains the spring 70 in a compressed condition applying a force to the forward surface 73 of the stem 32. When the mounting 21 and gripper device 43 are released, the spring 70 propels the gripper device 43 and mounting 21 attached thereto into the cavity 16.

The above described preferred embodiments has the advantage that upon completion of the injection stroke, the needle 12 is withdraw within the syringe 10 so as to not project outwardly from the cap 23. Accordingly, the probability of a needle stick injury occurring is reduced. A further advantage of the above described preferred embodiments is its ease of manufacture and reliability of operation relative to previous devices that also withdraw the needle into the interior of the piston rod.

A further advantage of the above described preferred embodiments, is the piston rod 15 and therefore needle 12 contained therein are captively located within the barrel 27 upon completion of the injection stroke.

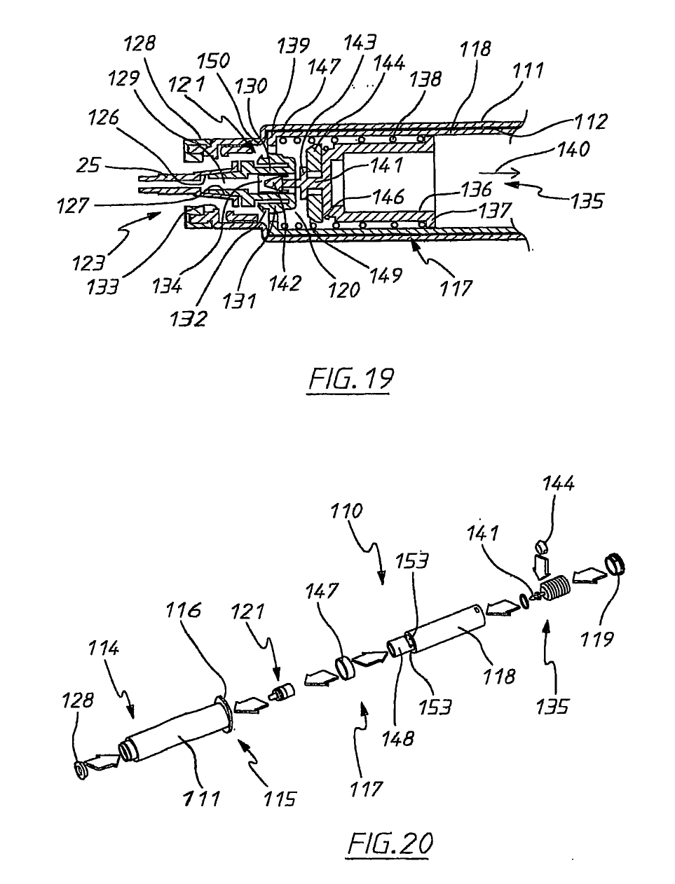

In the embodiment of FIGS. 18 to 20 the body 136 consists of a stem 152 terminating with a circular flange 153 against which the spring 138 presses due to the compression the spring 138 is subjected to. Surrounding the stem 152 is a seal 154 that performs the same task as the previously mentioned seal 147.

A further aspect in respect of this embodiment is the use of an annular clip 155 that extends between the forward portion of the stem 152 and the plunger 15. This annular clip 155 is radially expanded by engagement with the ramp surface 158 to thereby release the gripper device 135. The annular clip 155 has resilient fingers 156 that engage over a barb portion 157 so as to be fixed thereto when the gripper device 135 is released.

This is a similar operation to that of the syringe of FIG. 1. In this respect the annular catch 155 engages a ridge 158 of the gripper device 135.

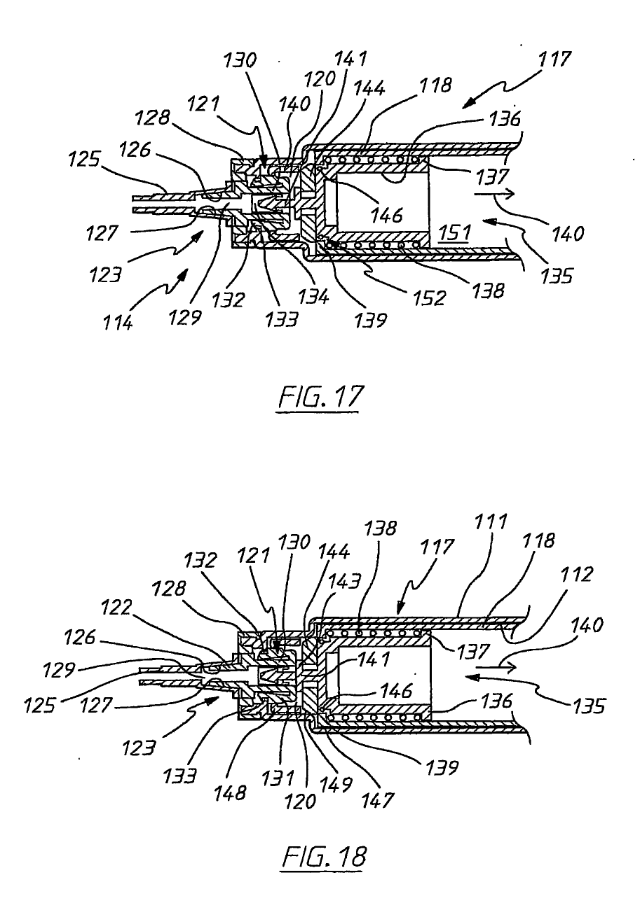

In the embodiment of FIGS. 15 to 17, the syringe 10 is modified relative to the syringe of FIGS. 18 to 20 in that the spring 138 extends through the substantial length of the plunger 15 and is tensioned. Upon the gripper device 135 being released, the spring 138 draws the gripper device back into the plunger 15. The spring 138 is attached to the rearward end of the stem 152 and the base 159 of the plunger 15.

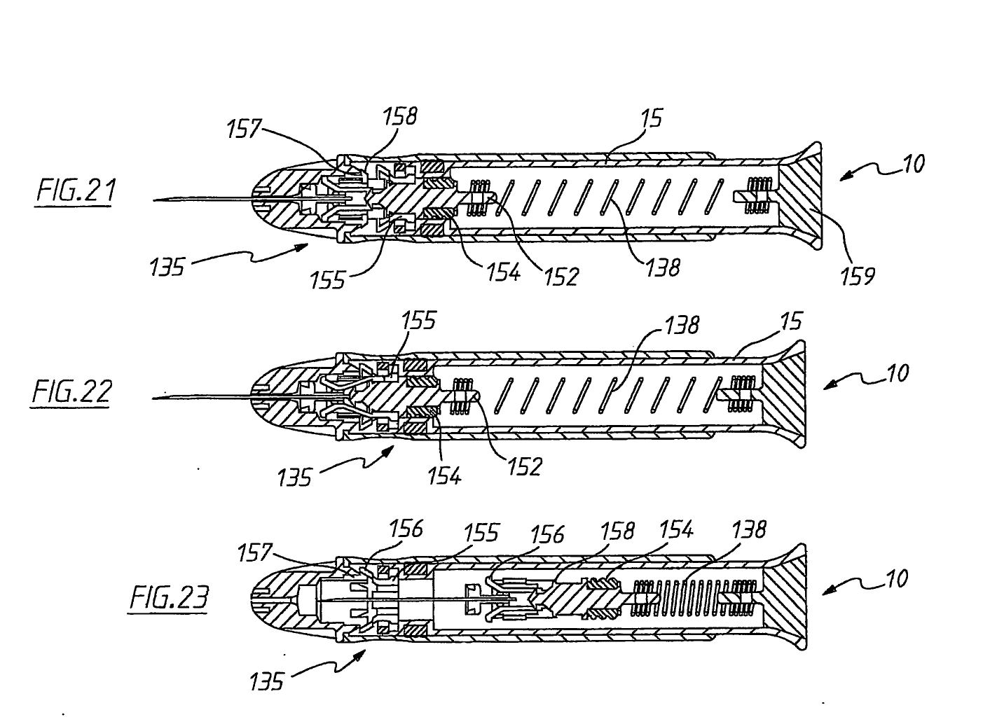

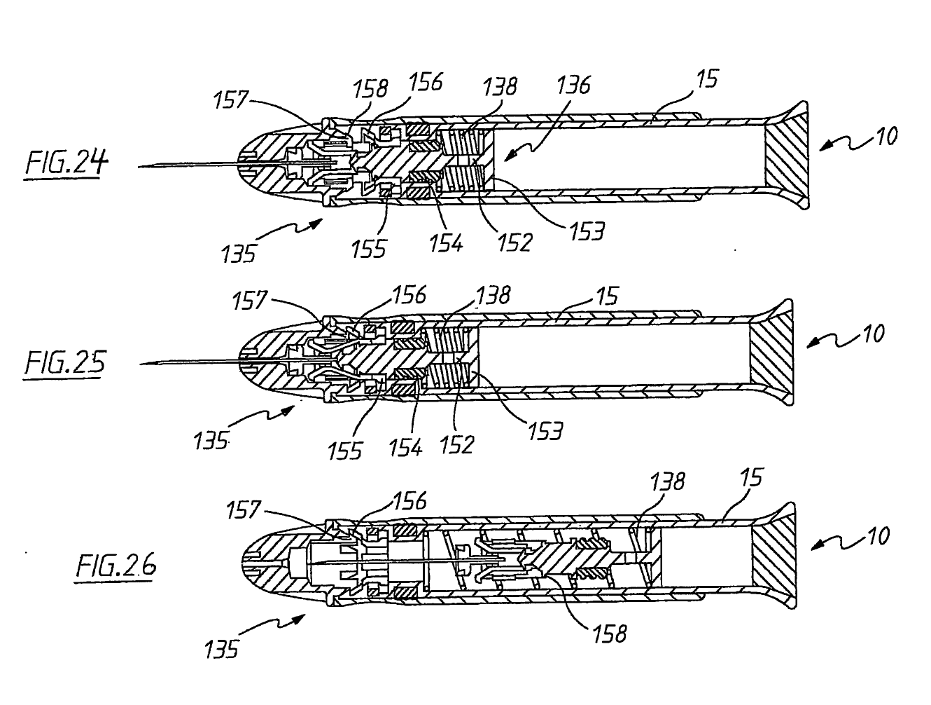

In FIGS. 21 to 26 of the accompanying drawings there is schematically depicted a syringe 110. The syringe 110 includes a barrel 111 with a generally cylindrical internal bore 112. The syringe 110 has a longitudinal axis 113, a forward end 114 and a rearward end 115. The rearward end of the barrel 111 is provided with a flange 116 that in a typical manner used by the operator to use the syringe 10.

Extending inwardly of the barrel 111 is a rod assembly 117 including a piston rod 118. The assembly 117 is located in the barrel 111 and extends outwardly from the rear end 115 so that a user may engage the end plug 119 to operate the syringe 110. The rod 118 slidably engages the bore 112 so as to enclose a chamber 120 between the assembly 117 and forward end 117.

Closing the forward end of the bore 112 is a needle mounting 121 having a forwardly projecting conical connector 122 that engages a needle assembly 123 including a needle 124 and connector 125. The connector 125 has a conical internal surface 126 that frictionally engages the conical surface 127 of the connector 122.

The mounting 121 slidably engages a seal 28 that co-operates with the mounting 121 to close the forward end of the chamber 120.

The mounting 121 has a longitudinal passage 129 through liquid passes to be delivered to the needle 124 to be injected.

The mounting 121 further includes a rearwardly extending generally cylindrical portion 130 that surrounds the passage 129 and supports a plurality of resilient fingers 131. The fingers 131 each have a recess 132 that engages an annular lip 133 of the barrel 111.

The portion 130 includes a plurality of apertures 134 adjacent which there is located a catch flange 135, which flanges 135 co-operate to provide a catch.

The abovementioned resilient fingers 131 provide a retaining means 150 in respect of the mounting 121, to retain the mounting 121 releasably attached to the barrel 111.

The rod 118 is hollow 20 as to provide a cavity 151 extending from a forward opening 152 of the rod 118 and movably supports a gripper device 135. The device 135 includes a body 136 that is contained within the rod 118 and includes a flange 137. A spring 138 is compressed against the flange 137 and a flange 139 of the rod 118 so that the body 136 is urged to move in the direction of the arrow 140. Projecting forwardly of the body 136 is a stem 141 terminating at its forward end with an enlarged portion 142. The stem 141 also has projecting from it a generally annular flange 143 that retains in position a gripper retainer 144. The retainer 144 is a “circlip” that is radially compressible relative to the axis 113 between a position (as shown in FIGS. 21 to 23) securing the device 130 to the forward end of the rod 118, and a radially compressed position (as shown in FIG. 24) releasing the assembly 135 for rearward movement, that is for movement in the direction of the arrow 140. The retainer 144 passes through openings 153.

The enlarged portion 142 is adapted to engage the catch flanges 145.

The device 135 also includes a seal 146 that sealingly connects the device 135 to the flange 139 of the rod 118 so as to sealing close the chamber 120.

A further seal 147 sealingly connects the rod 118 with the bore 112.

Attached to and projecting forwardly of the rod 118 is an actuator 148 that is in the form of a generally annular flange having an inclined leaning surface 149. The openings 153 are located between the rod 118 and actuator 148.

In operation of the above described syringe 110, the assembly 123 is moved rearwardly so that liquid is drawn into the chamber 120. When a liquid is to be injected, the assembly 123 is moved forwardly toward the end 114 so that the volume of the chamber 120 is reduced. The liquid to be injected is forced through the passage 129 to be ultimately delivered via the needle 124.

Toward the end of the stroke of the assembly 123, the gripper device 135 engages the mounting 121. More particularly the enlarged portion 142 of the stem 141 engages the catch flanges 146 of the mounting 121 so as to be fixed thereto. Thereafter further forward movement of the assembly 123 causes the ramp surface 149 to engage the fingers 131 to cause them to move radially inwardly, thereby being released from the lip 133. Accordingly the mounting 121 is free to move with the body 136. Still further forward movement of the assembly 123 causes radial compression of the clip 144 by engagement of the inclined surface 149 of the clip 144 with the inclined surface 150 of the barrel 111. When the clip 144 is sufficiently radially compressed the body 134 is released from the rod 118 and is propelled rearwardly in the direction of the arrow 140 by the spring 138. However as the mounting 121 is fixed to the body 136, the mounting 121 and the needle 124 attach thereto are drawn inwardly of the rod 118 so as to be contained within the cavity 151.

1. A syringe having a longitudinal axis, a forward end with a needle, and a rearward end, said syringe including:
a barrel providing a cylindrical bore;
a needle mounting to which said needle is fixed so as to extend forwardly therefrom;
a piston rod assembly slidably received in said bore and in sealing contact therewith so as to co-operate with said bore to provide a variable volume chamber to receive a liquid to be injected, said assembly including;
a hollow rod extending rearwardly from within said barrel to enable a user to move said assembly to vary said volume, said rod having a cavity extending rearwardly from a forward opening in said rod;
a gripper device mounted at said forward opening;
said syringe further including:
a gripper retainer extending between the device and rod to maintain said device fixed to said rod and movable to release said device so that said device moves into said cavity upon said gripper retainer moving forward at said fore end; and wherein
said mounting closes said chamber with said needle communicating therewith so that upon a reduction in volume of said chamber said liquid is forced through said needle, said mounting including:
a body engaged by said gripper device when adjacent said forward end and before said retainer releases said device, said body when engaged by said device is fixed thereto;
an element for urging said body and device in to said cavity; and wherein
said mounting includes a mounting retainer securing said mounting to said barrel but operable to release said mounting so that said mounting moves together with said needle, with said device into said cavity, said mounting retainer being radially moved inward from a retaining position to a release position by forward movement of said piston rod; and
an actuation member moved longitudinally forward by the forward movement of said piston rod to thereby actuate said mounting retainer to move radially inward to release said mounting after engagement of said mounting with said device.

2. The syringe of claim 1, wherein the element for urging includes said gripper device closing said opening so that said cavity can maintain a reduced internal pressure relative to atmosphere, so that upon said device engaging said body and said device and mounting being released, said device mounting and needle are moved into said cavity.

3. The syringe of claim 1, wherein said element for urging includes a spring engaging said mounting and urging said mounting and device into said cavity.

4. The syringe of claim 1, wherein the gripper retainer engages a portion of said barrel at said forward end to release said device.

5. The syringe of claim 1, wherein said gripper retainer has an engaging portion that upon complete of the injection stroke attaches the rod to the barrel to prevent rearward movement of the rod.

6. The syringe of claim 1, wherein the mounting includes a rearwardly extending projection, and said gripper device includes a cavity to receive said projection with said projection and gripper device engaging to captively locate said projection in said cavity.

7. The syringe of claim 6, wherein said gripper device includes a neck extending to said cavity, with said projection passing through said neck, with said projection including an expansion member that contracts as projection passes through said neck and expands to captively locate the projection in said cavity.

8. The syringe of claim 1, wherein said mounting includes a forward portion to sealingly connect the mounting to the barrel.

9. The syringe of claim 8, wherein said syringe includes a cap at the forward end of said barrel, which cap receives said mounting and said forward portion.

10. The syringe of claim 9, wherein said cap includes at least one passage to allow air to enter the cap so that air pressure is applied to the mounting to urge the mounting into the rod cavity.

11. The syringe of claim 1, wherein said actuator member is a sleeve surrounding said mounting.

12. The syringe of claim 11, wherein said mounting retainer includes a plurality of fingers that are resiliently urged outwardly, and are engaged by a ramp surface of said actuator member to be moved radially inwardly to release the mounting.

13. The syringe of claim 12, wherein said cap includes a forwardly facing abutment surface engaged by said fingers.

14. The syringe of claim 13,
wherein said means to urge includes a spring engaging said mounting and urging said mounting and device into said cavity,
wherein said mounting includes a forward portion to sealingly connect the mounting to the barrel,
wherein said syringe includes a cap at the forward end of said barrel, which cap receives said mounting and said forward portion, and
wherein said spring is located in said cap and engages said cap and mounting so as to be compressed therebetween to urge said mounting and device into said cavity.

15. A syringe having a longitudinal axis, a forward end to which a needle is to be attached, and a rearward end, said syringe including:
a barrel providing a cylindrical bore;
a needle mounting to which said needle is to be attached so as to extend forwardly therefrom, said mounting being at said forward end and providing for the delivery of a liquid to be injected to the needle;
a piston rod assembly slidably received in said bore and in sealing contact therewith so as to co-operate with said bore to provide a variable volume chamber to receive the liquid to be injected, the chamber being located between said rod assembly and said forward end, said piston rod assembly including:
a piston rod extending rearwardly from within said barrel to enable a user to move said assembly to vary said volume, said rod having a cavity extending rearwardly from a forward opening in said piston rod;
a gripper device mounted at said forward opening and having a stem projecting towards said forward end,
a resilient member located in said rod and attached to said rod and device and urging the device into said cavity,
a gripper retainer extending between the device and rod to maintain said device fixed to said rod and operable to release said device so that said device moves into said cavity upon said gripper retainer moving forward at said forward end; and wherein
said mounting closes said chamber so that upon a reduction in the volume of said chamber said liquid is forced through said needle, said mounting including:
a body engaged by said gripper device when adjacent said forward end and before said retainer releases said device, said body when engaged by said device being fixed thereto, said body having a passage into which said stem is to project to fix the body to the gripper device;
a mounting retainer securing said mounting to said barrel but operable to release said mounting so that said mounting moves, together with the needle when attached thereto, with said device into said cavity, said mounting retainer being moved inwardly relative to said axis from a retaining position to a release position by forward movement of said piston rod; and wherein
said syringe further includes:
an actuation member moved longitudinally forward by the forward movement of said piston rod to thereby actuate said mounting retainer to move inwardly to release said mounting after engagement of said mounting with said device.

16. The syringe of claim 15, wherein said resilient member is a spring urging said device into said cavity.

17. The syringe of claim 16, wherein said spring is compressed to urge the device into said cavity.

18. The syringe of claim 16, wherein said spring is tensioned towards the device into said cavity.

19. The syringe of claim 18, wherein said spring extends between a rearward portion of said device and a rearward portion of said plunger.

20. The syringe of claim 17, wherein said spring extends between a rearward portion of said device and a forward portion of said rod.

21. The syringe of claim 15, wherein said actuation member is part of said rod assembly.

22. The syringe of claim 21, wherein said actuation member is fixed to said rod.

23. The syringe of claim 15, wherein said mounting includes a forward portion sealingly connecting the mounting to the barrel.

24. The syringe of claim 15, wherein said actuation member is a sleeve surrounding said stem.

25. The syringe of claim 15, wherein said mounting retainer includes a plurality of fingers that are resiliently urged outwardly relative to said axis, and are moved inwardly relative to said axis to release said mounting.

26. The syringe of claim 15, wherein said mounting includes a catch to engage said stem when said stem projects into said mounting.

27. The syringe of claim 26, wherein said stem has an enlarged forward extremity that is engaged by said catch.

28. The syringe of claim 15, wherein said gripper retainer is moved inwardly with respect to said axis to release said device.

29. The syringe of claim 15, wherein said gripper retainer engages said barrel to be moved inwardly with respect to said axis.

30. The syringe of claim 24, wherein said gripper retainer is a circlip that is resiliently deformed by said barrel to be moved inwardly with respect to said axis.

31. The syringe of claim 15, wherein said gripper device projects inwardly of said rod and is slidably guided thereby for movement between a forward position to engage said mounting, and a retracted position located within said rod together with said mounting.
